(19) United States
(12) Patent Application Publication
(10) Pub. No.: US 2005/0026873 A1
(43) Pub. Date: Feb. 3, 2005

(54) Type-I methionyl aminopeptidases inhibitors in antibacterial targeting

(76) Inventors: Richard C. Holz, Providence, UT (US); Alicja J. Copik, Logan, UT (US); Krzysztof Swierczek, Logan, UT (US); Sabina Swierczek, Logan, UT (US)

Correspondence Address: John C. Stringham;WORKMAN NYDEGGER, 1000 Eagle Gate Tower, 60 East South Temple, Salt Lake City, UT, 84111, US

(21) Appl. No.: 10/786,240

(22) Filed: Feb. 25, 2004

Related U.S. Application Data
(60) Provisional application No. 60/491,074, filed on Jul. 30, 2003.

(51) Int. Cl. A61K 31/675; C07F 9/28; A61K 31/66; C07D 285/12

(52) U.S. Cl. 514/114; 514/363; 514/364; 514/365; 514/374; 514/383; 514/406; 548/136; 548/143; 548/200; 548/217; 548/253; 548/335.5; 548/364.1; 562/11

(57) ABSTRACT

Novel compounds act as molecular inhibitors to target new enzymatic targets on bacteria and provide a new class of bactericidal compounds. The compounds provide for selective inhibition of Type I methionyl aminopeptidases (MetAPs) in bacteria. Because humans and other animals have both type-I and type-II MetAPs, which are functionally equivalent, the compounds are bactericidal but do not completely inhibit MetAP activity in humans and other animals. The compounds also have anti-microbial activity in microbes other than bacteria provided the targeted microbes have predominately type-I MetAP.

### CROSS-REFERENCE TO RELATED APPLICATIONS

This application claims the benefit of U.S. Provisional Patent Application Ser. No. 60/491,074, filed Jul. 30, 2003, entitled “Type-I Methionyl Aminopeptidases Inhibitors in Antibacterial Targeting,” which is incorporated herein by reference.

### BACKGROUND OF THE INVENTION

1. The Field of the Invention

The present invention relates generally to compounds with pharmaceutical activity. More particularly, embodiments of the invention relate to pharmaceutically active compounds that are useful for the treatment of microbial and bacterial infections.

2. The Relevant Technology

Bacterial infections are a significant and growing medical problem in the United States and throughout the world. In part, this is because an increasing number of disease-causing microbes have become resistant to existing antibiotics. Tuberculosis, gonorrhea, malaria, and childhood ear infections are just a few of the diseases that have become hard to treat with antibiotic drugs.

In addition to its adverse effect on public health, antimicrobial resistance contributes to higher health care costs. Treating resistant infections often requires the use of more expensive or more toxic drugs and can result in longer hospital stays for infected patients. The Institute of Medicine, a part of the National Academy of Sciences, has estimated that the annual cost of treating antibiotic resistant infections in the United States may be as high as $30 billion.

Part of the problem is that bacteria and other microorganisms that cause infections are remarkably resilient and can develop ways to survive drugs meant to kill or weaken them. Microbes generally are unicellular creatures that, compared with multicellular organisms, have a small number of genes. Even a single random gene mutation can have a large impact on their disease-causing properties. Since most microbes replicate very rapidly, they can evolve rapidly. Thus, a mutation that helps a microbe survive in the presence of an antibiotic drug will quickly become predominant throughout the microbial population. Microbes also commonly acquire genes, including those encoding for resistance, by direct transfer from members of their own species or from unrelated microbes.

This antibiotic resistance, also known as antimicrobial resistance or drug resistance, is due largely to the increasing use of antibiotics. One prevalent cause of the problem is that antibiotics are given to patients more often than guidelines set by federal and other healthcare organizations recommend. For example, patients sometimes ask their doctors for antibiotics for a cold, cough, or the flu, all of which are viral and don't respond to antibiotics. Also, patients who are prescribed antibiotics but don't take the full dosing regimen can contribute to resistance because they fail to kill off all the bacteria. Although steps are being taken to remedy, or at least reduce, this problem, an increase of antimicrobial resistance to current antibacterial drugs is ongoing.

Antibiotic resistance has been recognized since the introduction of penicillin nearly 50 years ago when penicillin-resistant infections caused by Staphylococcus aureus rapidly appeared. Today, hospitals worldwide are facing an unprecedented crisis from the rapid emergence and dissemination of other microbes resistant to one or more antimicrobial agents. In fact, many physicians are concerned that several bacterial infections soon may be untreatable.

For example, in the United States the Centers for Disease Control and Prevention (the CDC) have reported that several strains of Staphylococcus aureus are resistant to all antibiotics including the most powerful, vancomycin. Increasing reliance on vancomycin has led to the emergence of vancornycin-resistant enterococci (VRE), bacteria that infect wounds, the urinary tract and other sites. Until 1989, such resistance had not been reported in U.S. hospitals. By 1993, however, more than 10 percent of hospital-acquired enterococci infections reported to the CDC were resistant.

Other examples of microbial strains that have been reported to be developing drug resistance include Streptococcus pneumoniae, which causes thousands of cases of meningitis and pneumonia, and 7 million cases of ear infections in the United States each year; gonorrhea; malaria; various strains of multidrug-resistant tuberculosis (MDR-TB), which have emerged over the last decade and pose a particular threat to people infected with HIV; diarrheal diseases such as Shigella dysenteriae, Campylobacter, Vibrio cholerae, Escherichia coli and Salmonella, which cause almost 3 million deaths a year; and fungal pathogens, which are common among AIDS patients.

Currently, many of the broad spectrum of antibiotics, such as penicillins, cephalosporins, monobactams and the carbapenems, are heavily relied upon by doctors to treat common bacterial infections contain β-lactam functional units. β-lactam functional units target enzymes involved in bacterial cell wall synthesis or pathways involved in cell replication. Since any new β-lactam antibiotic will simply be a structural variant of an existing compound and target the same enzymatic pathways, it is likely that any new β-lactam drugs discovered will quickly become useless as bacteria easily mutate and develop resistance to the new drugs. Therefore, current avenues for the development of new antibiotic drugs offer diminishing returns.

Accordingly, it would represent an advance in the art if there were new compounds that exhibited antibacterial activity without relying on β-lactam functional units. Ideally, such new compounds would target enzymes involved in bacterial cell wall synthesis or pathways involved in cell replication.

### SUMMARY OF EMBODIMENTS OF THE INVENTION

In general, embodiments of the present invention are concerned with new compounds that act as molecular inhibitors to target new enzymatic pathways on bacteria. These new compounds provide a new class of bactericidal compounds that have potential for new antibacterial drugs. Generally, these novel compounds provide for selective inhibition of methionyl aminopeptidases (MetAPs) in bacteria. Additionally, the present invention also relates to methods for treating subjects by selectively inhibiting type-I MetAP.

In particular, MetAP activity can be selectively inhibited in bacteria instead of humans or other animals because bacteria have only type-I MetAP whereas humans and other animals have both type-I and type-II MetAPs. Although type-I and type-II MetAPs are nearly structurally identical, one key difference has been identified and can be selectively inhibited. This difference is that active site cysteine residue (e.g. C59 and C70 in E. coli) exists only in type-I MetAPs. Thus, type-I MetAPs that contain C59 or C70 are viable targets for the design of a new class of antibacterial agent as described and claimed herein.

According to one embodiment of the invention, the following non-limiting set of exemplary compounds have been designed as type-I MetAP inhibitors: 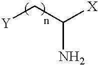
wherein: X is selected from the group consisting of CH₂SH, CH₂OH, NHOH, PO₃H₂, pyrazoles, imidazoles, oxazoles, isoxazoles, thiazoles, isothiazoles, triazoles, oxadiazoles and thiadiazoles; and Y is selected from the group consisting of: COCZ, C(EWG)Z, SOCZ, SO₂CZ, 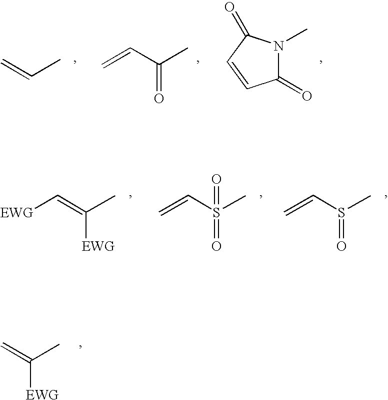
and pharmaceutically acceptable salts thereof, wherein: Z is selected from the group consisting of chlorine, bromine, and iodine; EWG is an electron withdrawing group selected from the group consisting of CHO, COR, COOH, COOR, NO₂, CN, SOR, SO₂R, SO2OR and R can be any alkyl or aryl group, examples of which include: methyl, ethyl, propyl, i-propyl, butyl, s-butyl, t-butyl, phenyl, substituted phenyl, naphthyl, substituted naphthyland n is an integer, preferably four or five.

Another embodiment of the invention includes a set of compounds that have a formula selected from the group consisting of: 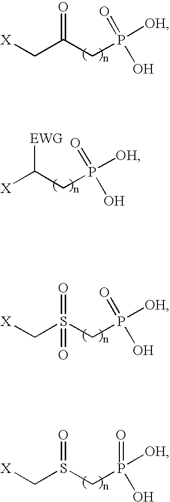
and pharmaceutically acceptable salts thereof, wherein X is chlorine, bromine, or iodine; EWG is an electron withdrawing group selected from the group consisting of CHO, COR, COOH, COOR, NO₂, CN, SOR, SO₂R, and SO₂OR; R can be any alkyl or aryl group, examples of which include: methyl, ethyl, propyl, i-propyl, butyl, s-butyl, t-butyl, phenyl, substituted phenyl, naphthyl, substituted naphthyl; and n is an integer, preferably four or five.

Yet another embodiment of the invention includes a set of example compounds having a formula selected from the group consisting of: 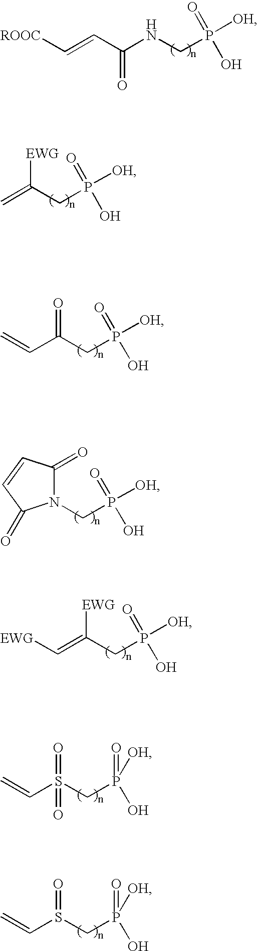
and pharmaceutically acceptable salts thereof, wherein: EWG is an electron withdrawing group selected from the group consisting of CHO, COR, COOH, COOR, NO₂, CN, SOR, SO₂R, SO₂OR; R can be any alkyl or aryl group, examples of which include: methyl, ethyl, propyl, i-propyl, butyl, s-butyl, t-butyl, phenyl, substituted phenyl, naphthyl, substituted naphthyl; and n is an integer, preferably four or five.

According to another embodiment of the invention, a method of providing an antibacterial dosage to a subject in need thereof includes administering to a subject an effective amount of a compound that is selectively configured to inhibit Type-I MetAP, the compound comprising the formula:
A-B-C
wherein: A is a functional group selected to covalently bond with a recognition site on Type-I MetAP, such as Cys 59 or C70 (E. coli numbering scheme); C is an electrophilic functional group selected to inhibit a catalytic site on Type-I MetAP; and B comprises a series of one or more groups (e.g. a four or five carbon chain) selected to separate A and C such that each of A and C effectively bind to the respective recognition and active sites on Type-I MetAP.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims.

### BRIEF DESCRIPTION OF THE DRAWINGS

In order that the manner in which the above-recited and other advantages and features of the invention are obtained, a more particular description of the invention briefly described above will be rendered by reference to specific embodiments thereof which are illustrated in the appended drawings. Understanding that these drawings depict only typical embodiments of the invention and are not therefore to be considered limiting of its scope, the invention will be described and explained with additional specificity and detail through the use of the accompanying drawings in which:

FIG. 1 is a chart that illustrates the classifications of MetAPs;

FIG. 2 is a molecular diagram that illustrates the active site of EcMetAP-I;

FIG. 3 is a molecular diagram that illustrates the binding mode of three phosphonate inhibitors of EcMetAP-I;

FIG. 4 is a molecular diagram that illustrates the residues making up the S1 and S2 substrate binding pockets in EcMetAP-I;

FIG. 5 is a molecular diagram that illustrates important functional groups on the peptide substrate for MetAPs;

FIG. 6 is a molecular diagram that illustrates a covalent molecular inhibitor of EcMetAP-I;

FIG. 7 is a molecular diagram that illustrates a covalent molecular inhibitor of EcMetAP-I;

FIG. 8 is a chart depicting the modification of EcMetAP-I and PfMetAP-II by DTNB;

FIG. 9 is a chart depicting the modification of C59A EcMetAP-I and C70A EcMetAP-I by DTNB; and

FIG. 10 is a chart illustrating the reaction of two potential covalent molecular inhibitor with various MetAPs.

### DETAILED DESCRIPTION OF THE INVENTION

The present invention relates to new compounds that act as molecular inhibitors to target new enzymatic targets within bacteria. These new compounds provide a new class of bactericidal compounds that have potential for new antibacterial drugs. Generally, these novel compounds provide for selective inhibition of methionyl aminopeptidases (MetAPs) in bacteria. Additionally, the present invention also relates to methods for treating subjects by selectively inhibiting type-I MetAP.

Embodiments of the present invention also provide for inhibition of type-I MetAP in microbes other than bacteria, for example Fungi and Leashmania. Those skilled in the art will understand, in view of the disclosure herein, how the inhibition of type-I MetAP in microbes other than bacteria can be obtained.

In particular, MetAP activity can be selectively inhibited in bacteria without being completely stopped in humans or other animals because bacteria have only type-I MetAP whereas humans and other animals have both type-I and type-II MetAPs. Although type-I and type-II MetAPs are nearly structurally identical, one key difference has been identified that can be exploited. This difference is a single active site cysteine residues (e.g. C59 or C70 in E. coli) that exist only in type-I MetAPs and can be selectively inhibited by targeting. Thus, type-I MetAPs that contain C59, for example, are viable targets for the design of a new class of antibacterial agent as described and claimed herein.

Although the side effects are still unknown, it is anticipated that because human cells have both type-I and type-II enzymes, the side effects will be minimal due to the overlapping function in eukaryotic cells of the type-I and type-II enzymes. Thus, even though the type-I enzymes may be inactivated in humans, the type-II enzymes can offer a substitute pathway to carry out the same function to minimize side effects.

Reference will now be made to the drawings which illustrate various aspects of exemplary embodiments of the invention. It is to be understood that the drawings are diagrammatic and schematic representations of such exemplary embodiments, and are not limiting to the present invention, nor are they necessarily drawn to scale.

In the following description, numerous specific details are set forth in order to provide a thorough understanding of the present invention. It will be obvious, however, to one skilled in the art that the present invention may be practiced without these specific details. In other instances, well-known aspects of pharmaceutical compounds and methods have not been described in particular detail in order to avoid unnecessarily obscuring the present invention.

As used herein, the term “subject” denotes any human or non-human animal. Non-human animals include, e.g., mammals, birds, reptiles, amphibians and fish. Preferably, the non-human animal is a mammal, e.g., a mouse, a rat, a rabbit, a monkey, a dog, a cat, a pig, a cow, a horse, or a sheep.

The present invention also includes pharmaceutically acceptable salts of the antibacterial compounds of the invention. A “pharmaceutically acceptable salt” includes a salt that retains the desired biological activity of the parent compound and does not impart any undesired toxicological effects. Examples of such salts are salts formed by reacting or complexing the inventive compounds with acids such as hydrochloric acid, hydrobromic acid, sulfuric acid, phosphoric acid, nitric acid, and the like; acetic acid, oxalic acid, tartaric acid, succinic acid, malic acid, benzoic acid, pamoic acid, alginic acid, methanesulfonic acid, naphthalenesulfonic acid, and the like. Also included are salts of cations such as sodium, potassium, lithium, zinc, copper, barium, bismuth, calcium, and the like; or organic cations such as trialkylammonium. Combinations of the above salts are also included.

Generally, the pharmaceutical compositions of the invention can be used to treat infections by gram-positive bacteria and gram-negative bacteria having a Cys residues in its MetAP. Accordingly, as used herein for simplicity, unless otherwise indicated, the term “bacterial infection(s)” includes bacterial infections that occur in a subject as well as disorders related to bacterial infections that may be treated or prevented by administering antibacterials such as the compounds of the present invention. Such bacterial infections, and disorders related to such infections, include, by way of example only, the following: Staphylococcus aureus, E. Coli, tuberculosis, Brucellosis, Campylobacteriosis, Cholera, Glanders (Burkholderia mallei), Leprosy, Ulcers (Helicobacter pylori), Ehrlichiosis, Leptospirosis, Melioidosis (Whitmore's disease), Rocky Mountain spotted fever, Q fever (Coxiella burnetii), Shigellosis, Tularemia, Typhoid fever, Vibrio parahaemolyticus, Vibrio vulnificus, and Yersiniosis.

I. Methionyl Aminopeptidases

Methionyl aminopeptidases (MetAPs) represent a unique class of protease that is capable of removing N-terminal methionine residues from nascent polypeptide chains. Since the biosynthesis of all prokaryotic and eukaryotic (eukaryotes include all organisms except archaea, viruses, bacteria, and blue-green algae) proteins present in the cytosol starts with the initiator amino acid methionine, MetAPs play a central role in protein synthesis. The physiological importance of MetAP activity is underscored by the cellular lethality upon deletion of the MetAP gene in Escherichia coli, Salmonella typhimurium and Saccharomyces cerevisiae.

While the rationale for the removal of the initiator methionine remains unclear, several explanations have been proposed. Some involve the facilitation of further processing after excision of the N-terminal group such as removal of signal sequences if present, proteolytic cleavage to generate shorter peptides and the covalent attachment of residues and blocking groups such as acetyl and myristoyl groups. The structure of the mature N-terminus plays important roles in N-directed degradation pathways and in targeting to cellular membranes. MetAPs are therefore one of the key cellular enzymes involved in protein maturation. Recently, it was shown that MetAPs are the target for the sesquiterpene epoxide-containing anti-angiogenesis agents, ovalicin and fumagilin, one of which is currently in phase III clinical trials.

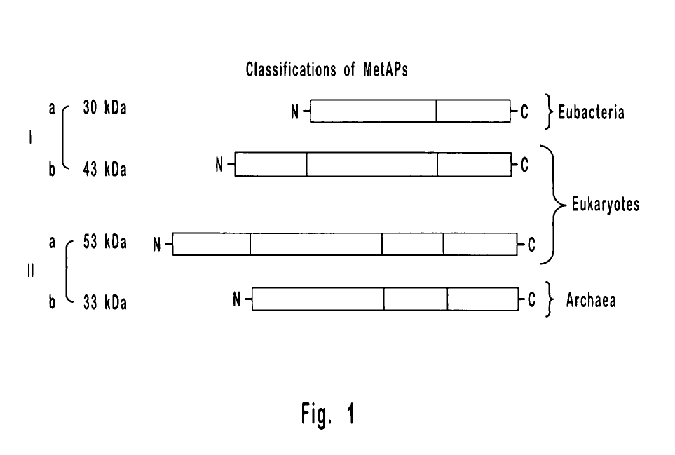

As illustrated in FIG. 1, MetAPs are organized into two classes, type-I and type-II. This classification is based on the absence or presence of an extra 62 amino acid sequence (of unknown function) inserted near the catalytic domain. Bacteria have only the type-I enzyme while archaea has only the type-II enzyme. Eukaryotic cells, in contrast, have both type-I and type-II enzymes. Because each of the type-I and type-II enzymes provide the same function, there is redundancy in eukaryotic cells in that each of the type-I and type-II enzymes can perform the same function even if one of them is absent or disabled.

These two classes are further subdivided based upon the presence of an additional N-terminal domain that has been found only in eukaryotic MetAPs (Type-Ib & Imb). The N-terminal region of yeast MetAP-I has sequences consistent with two zinc-finger structures. The porcine enzyme possesses an N-terminal domain similar in size to the yeast protein; however, it contains substantial stretches of polybasic acidic amino acids in lieu of the zinc-finger domains. Two basic polylysine blocks and one aspartic acid block have also been found in the N-terminal region of HsMetAP-II. These sequences have been proposed to form recognition/binding motifs for ribosomal attachment in juxtaposition to the nascent polypeptide chain, leading to the cotranslational removal of the initiator methionine.

It has been shown that S. cerevisiae (yeast) is viable if the gene encoding for the type-I MetAP is deleted but the type-II gene is present. Therefore, redundancy exists between type-I and type-II MetAPs. Since bacteria only contain type-I MetAPs while all human cells contain both type-I and type-II MetAPs, type-I MetAPs represent a novel antibiotic target if type-I MetAPs can be specifically targeted over type-II.

All MetAPs appear to have similar substrate specificities that are dictated by the penultimate residue. The amino acid residue adjacent to the N-terminal methionine must be physically small and/or uncharged (eg. Gly, Ala, Pro, Ser, Thr, Cys and Val). MetAPs do not remove methionine from substrates having a bulky or charged residue in the penultimate position, and the substrate preference is opposite to the “N-end rule” for protein degradation. MetAP will not remove an N-terminal methionine if a “destabilizing” amino acid will be exposed but larger amino acids at the N-terminus are recognized by an ubiquitin ligase and are polyubiquinated and degraded. On the other hand, retained initiator methionine has been postulated to act as a prophylactic cap, preventing premature degradation of proteins. Furthermore, residues downstream of the specificity-determining-residue were initially proposed to have little impact on the reaction; however, when longer octapeptide substrates were used, the values obtained for the Michaelis-Menten constant (Kₘ) were observed to be orders of magnitude lower than when three- and four-residue substrates were used. These data suggest that sequence-independent backbone interactions between substrate and enzyme likely exist.

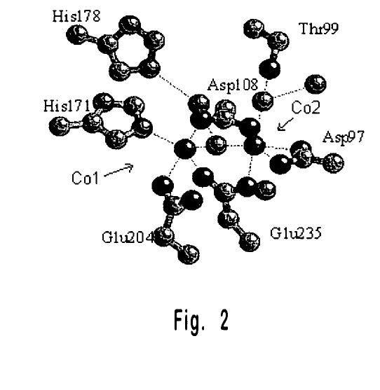

In addition, the MetAPs from E. coli (EcMetAP), Homo sapiens (HsMetAP), Staphylococcus aureus (SaMetAP), and Pyrococcus furiosus (PJMetAP) have been crystallographically characterized and all four display a novel “pita-bread” fold with an internal pseudo two-fold symmetry that structurally relates the first and the second halves of the polypeptide chain. Each of the halves contains an antiparallel β-pleated sheet flanked by two helical segments and a C-terminal loop. Both domains contribute conserved residues to form the metalloactive site. In all three structures a bis(μ-carboxylato)(μ-aquo/hydroxo)dicobalt core was observed with an additional carboxylate residue at each metal site and a single histidine bound to Co1 (FIG. 2).

Within the active site of all MetAPs, three solvent molecules are present that may play a role in catalysis (FIG. 2). The first water molecule bridges between the two cobalt ions while the second is terminally ligated to Co2 and piparticipates in a hydrogen bonding interaction with Thr99. A third solvent molecule bridges between an active site histidine residue, H178, and the aquo/hydroxo group bridging between the two Co(II) ions. This hydrogen-bonding network has been proposed to facilitate the formation of the nucleophile in the catalytic reaction. H178 is in close proximity to the cobalt ions (5.1 Å from Co1 and 6.6 Å from Co2), and is strictly conserved.

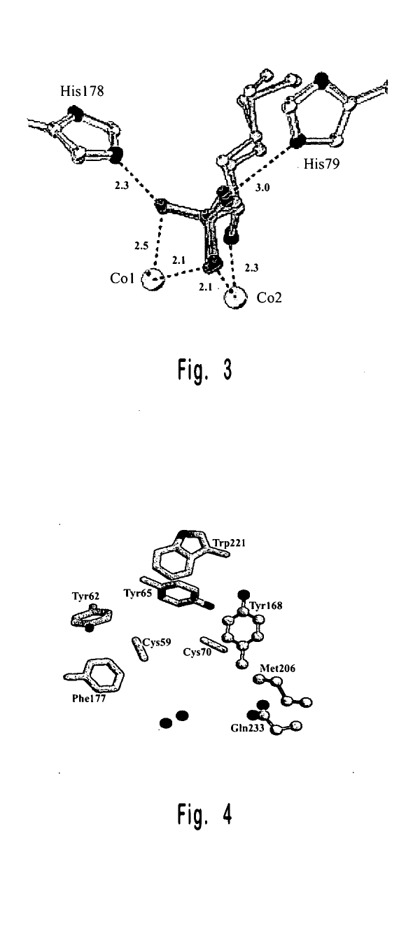

A catalytic mechanism for EcMetAP-I has been proposed in the prior art on the basis of X-ray crystallographic studies and molecular modeling. In this mechanism, the substrate binding step was based on an X-ray crystal structure of a substrate-like inhibitor ((3R)-amino-(2S)-hydroxyheptanoyl-L-Ala-L-Leu-L-Val-1-Phe-OMe) bound to EcMetAP-I which indicated that the N-terminus of the inhibitor was coordinated to Co2, the keto oxygen (analogous to the carbonyl carbon in a terminal peptide bond) was ligated to Co1 and the (2S)-hydroxyl group had displaced the bridging hydroxide ion. Glu204, which is bound in a monodentate fashion to the histidine-ligated cobalt ion (Co1), is proposed to act as a general base and proton shuttle. Abstraction of a proton from the bridging water/hydroxide molecule by Glu204 generates the nucleophile. Following attack on the carbon atom of the scissile peptide linkage, the tetrahedral transition-state is formed. The N-terminal amine group is coordinated to the second cobalt ion (Co2) while the gem-diolate intermediate remains in a bidentate coordination mode to Co1. This structure was suggested based on a recent X-ray crystal structure of a transition-state analog inhibitor, methionine phosphonate (L-MetP) bound to EcMetAP-I (FIG. 3). This structure revealed that the phosphonate group interacts in a bridging manner with Co1 and Co2 and that a second oxygen atom on the phosphonate group forms a hydrogen bond with H178. Proton donation by Glu204 to the leaving group amino nitrogen was proposed to cause the collapse of the transition-state into the corresponding products. While His79 is proposed to assist in positioning the substrate by hydrogen bonding to the amine group of the peptide linkage, His178 is proposed to stabilize the oxyanion transition-state via hydrogen bonding. No definitive role has been proposed for the second metal ion in this mechanism other than coordinating the amino-terminal group.

Two residues that are highly conserved in type-I MetAPs, but are absent in all type-II MetAPs, are the cysteine residues (EcMetAP-I: C59 and C70) that reside at the back of the substrate recognition pocket (FIG. 4). These Cys residues are 4.4 Å apart and, therefore, do not form a disulfide bond. Prokaryotes contain only type-I MetAPs whereas eukaryotes contain both type-I and type-II MetAPs. These two Cys residues therefore provide a potential target to differentiate type-I MetAPs from type-II MetAPs. Since bacteria only contains type-I MetAPs while all human and other animal cells contain both type-I and type-II MetAPs, selective inhibition of type-I MetAPs by targeting the cysteine residues C59 and C70 represents a novel antibiotic.

II. Antibacterial Compounds

The present invention encompasses a series of small molecules that have been designed based on molecular modeling studies to target and inhibit the activity of Type-I MetAP by targeting the C59 or C70 residue. Conceptually, these molecules can be considered to have three main parts: an active site inhibition functional group that binds to the metal center on MetAPs, a connection chain such as a four or five carbon chain, and an electrophilic substrate recognition functional group. These small molecules mimic the shape of methionine in order to provide the necessary shape recognition and covalently bond to type-I MetAP and inhibit the activity thereof. It is anticipated that compounds that target C59 (E. Coli numbering) will not inhibit HsMetAP-I or HsMetAP-II since neither enzyme contains this Cys residue, as indicated in the following table (wherein the letter “C” identifies the Cys residue). Therefore, one would predict that such compounds would have little or now side-effects. On the other hand, compounds that target C70, will potentially inhibit HsMetAP-I but not HsMetAP-II. Therefore, some side effects may be observed but because of cellular redundancy between type-I and -II MetAPs, it is anticipated that HsMetAP-II will be able to take over the function of cellular N-terminal methionine cleavage, thereby minimizing toxic side effects.

Amino Acid Sequence Alignment of Selected type -I
and -II MetAPs

| | | |
|---|---|---|
| | | 59 70 |
| | | Ec C L G Y H G Y P K S V C I S I |
| | | Hi C L N Y H G F P K A T C I S I |
| Type-I | {open oversize brace} | St C L G Y H G Y P K S V C I S I |
| | | Hp F K G L Y G F P N S V C M S L |
| | | Sc1 P L N Y Y N F P K S L C T S V |
| | | Hs1 P L N Y Y N F P K S C C T S V |
| | | Pf F - - - - - - - - P V N L S I |
| Type-II | {open oversize brace} | Sc2 F - - - - - - - - P T G L S L |
| | | Hs2 F - - - - - - - - P T G C S L |

(Ec) E. coli-I; (Hi) H. influenzae-I; (St) S. typhimurium-I. (All are Enterobacteria)
(Hp) H. pylori-I; (Sc) S. cerevisiae-I; (Hs) Homo sapiens-I.
(Pf) P. furiosus-II; (Sc) S. cerevisiae-II; (Hs) Homo sapiens-II.

For the active site inhibition functional group, phosphonic acid derivatives of L-methionine have been shown to be weak competitive inhibitors of EcMetAP-I. Therefore, phosphonic acid groups (PO₃H₂) will bind to the active site metal ion in EcMetAP-I. Still, although in-vivo studies have not been performed, it is theorized that highly charged functional groups such as phosphonic acid groups may have trouble crossing the cellular membrane. Accordingly, other effective functional groups according to the invention may include, by way of example only and not limitation, pyrazoles, imidazoles, and triazoles, and other nitrogen-containing molecules.

In addition, an amine group can be included on this side of the molecule to increase the effectiveness of the bond by attaching to adjacent recognition sites.

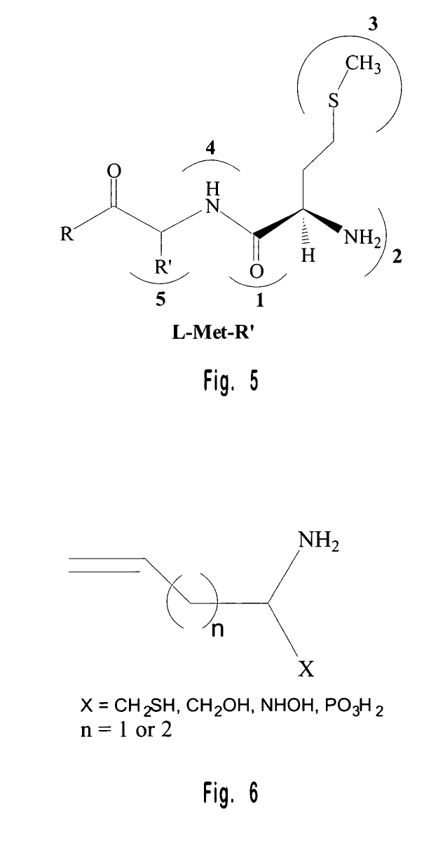

The intermediate chain, preferably carbon, provides the necessary molecular shape and size for site recognition. The carbon chain length can be adjusted to position the active site inhibition group (e.g. the phosphonic acid group) in order to maximize the metal binding affinity while also providing the correct chain length to position the electrophilic carbon center adjacent to C59 or C70. In addition, an N-terminal amine moiety can be configured to take into account recognition site 2 (FIG. 5) as well as other metal binding functionality's such as thiols, alkoxides, and hydroxymates (FIG. 6).

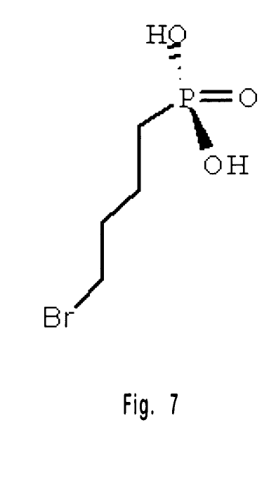

Based on the X-ray crystallographic data known in the prior art, on various inhibited forms of EcMetAP-I, side-chain lengths of 4 and 5 appear to fit most efficiently in the binding pocket. For a more in depth discussion, reference is made to Lowther, T. W., Zhang, Y., Sampson, P. B., Honek, J. F., and Matthews, B. W. “Insights into the Mechanism of E. Coli Methionine Aminopeptidase from the Structural Analysis of Reaction Products and Phosphorous-Based Transition State Analogs.” (1999) Biochemistry 38, 14810. For example, the test compound depicted in FIG. 7 has been shown to inhibit EcMetAP-I.

One aspect that must be considered in selecting the length of the sintermediate chain is the size of the other functional groups in the molecule. For example, a triazole with three nitrogens may already provide a two-carbon chain, thus reducing the size requirements for the intermediate carbon chain to a two to four carbon chain.

Finally, regarding the substrate recognition site, both Cys59 and Cys70 must be reduced for EcMetAP-I to be fully active, suggesting that these Cys residues must be reasonable nucleophiles. Therefore, the herein disclosed molecules have an electrophilic carbon center that is positioned adjacent to Cys59 and Cys70 so that one or both Cys residues can nucleophilically attack the inhibitor side-chain, thereby covalently inhibiting EcMetAP-I. Because these Cys residues are not present in type-II MetAPs, these molecules will likely be weak competitive inhibitors.

Examples of electrophilic active groups are depicted herein below in the example compounds. For example, it has been determined that iodine is a preferred member of an electrophilic active group.

In constructing antibacterial compounds to inhibit Type-I MetAP activity, it is important not only that the compounds work in theory or even in a Petri dish, but also that the compounds be compatible for consumption by a subject. For example, certain functional groups may be effective for selective Type-I MetAP inhibition yet negatively affect other physiological processes or simply be incompatible for uptake through the digestive system. For example, if the electrophilic group is too strongly electrophilic, it may bind to a histamine site (e.g. histamine 79 in E. coli) that is common to both type-I and type-II MetAP enzymes and consequently deactivate both type-I and type-II MetAP enzymes.

With the foregoing in mind, the following non-limiting set of example compounds have been designed as type-I MetAP inhibitors:

Another set of compounds that have been designed have a formula selected from the group consisting of: 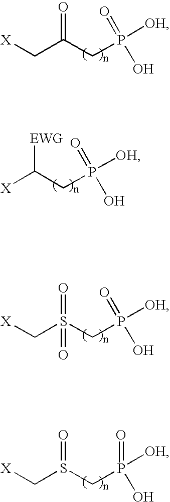
and pharmaceutically acceptable salts thereof, wherein X is chlorine, bromine, or iodine; EWG is an electron withdrawing group selected from the group consisting of CHO, COR, COOH, COOR, NO₂, CN, SOR, SO₂R, and SO₂OR; R can be any alkyl or aryl group, examples of which include: methyl, ethyl, propyl, i-propyl, butyl, s-butyl, t- 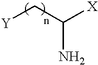
wherein: X is selected from the group consisting of CH₂SH, CH₂OH, NHOH, PO₃H₂, pyrazoles, imidazoles, oxazoles, isoxazoles, thiazoles, isothiazoles, triazoles, oxadiazoles and thiadiazoles; and Y is selected from the group consisting of: COCZ, C(EWG)Z, SOCZ, SO₂CZ, 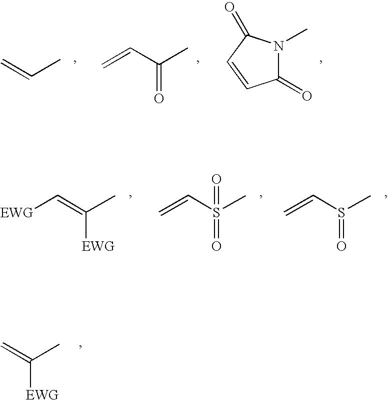
and pharmaceutically acceptable salts thereof, wherein: EWG is an electron withdrawing group selected from the group consisting of CHO, COR, COOH, COOR, NO₂, CN, SOR, SO₂R, and SO₂OR; Z is selected from the group consisting of chlorine, bromine, and iodine; R can be any alkyl or aryl group, examples of which include: methyl, ethyl, propyl, i-propyl, butyl, s-butyl, t-butyl, phenyl, substituted phenyl, naphthyl, substituted naphthyl; and n is an integer, preferably four or five.
butyl, phenyl, substituted phenyl, naphthyl, substituted naphthyl; and n is an integer, preferably four or five.

Yet another set of example compounds have a formula selected from the group consisting of: 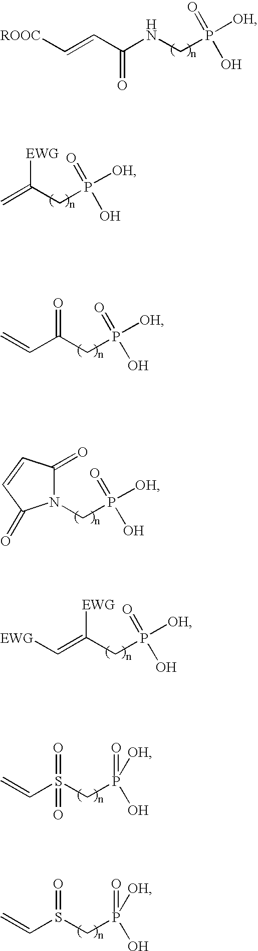
and pharmaceutically acceptable salts thereof, wherein: EWG is an electron withdrawing group selected from the group consisting of CHO, COR, COOH, COOR, NO₂, CN, SOR, SO₂R, and SO₂OR; R can be any alkyl or aryl group, examples of which include: methyl, ethyl, propyl, i-propyl, butyl, s-butyl, t-butyl, phenyl, substituted phenyl, naphthyl, substituted naphthyl; and n is an integer, preferably four or five.

Methods for forming the foregoing molecules are readily known to those skilled in the art. For example, see Example 9 hereinbelow.

III. Pharmaceutical Compositions

As used herein, the term “administering” to a subject includes dispensing, delivering or applying an antibacterial compound, e.g., an antibacterial compound in a pharmaceutical formulation (as described herein), to a subject by any suitable route for delivery of the compound to the desired location in the subject, including delivery by either the parenteral or oral route, intramuscular injection, subcutaneous/intradermal injection, intravenous injection, buccal administration, transdermal delivery and administration by the rectal, colonic, vaginal, intranasal or respiratory tract route.

As used herein, the term “effective amount” includes an amount effective, at dosages and for periods of time necessary, to achieve the desired result, e.g., sufficient to treat a bacterial infection in a subject without killing or unduly harming the subject. An effective amount of an antibacterial compound, as defined herein may vary according to factors such as the disease state, age, and weight of the subject, and the ability of the antibacterial compound to elicit a desired response in the subject. Dosage regimens may be adjusted to provide the optimum therapeutic response. An effective amount is also one in which any toxic or detrimental effects (e.g., side effects) of the antibacterial compound are outweighed by the therapeutically beneficial effects.

A therapeutically effective amount of an antibacterial compound (i.e., an effective dosage) will vary depending upon a variety of factors unique to each compound. A desired dosage will strike an equilibrium to kill bacteria by deactivating Type-I MetAPs while not deactivating Type-II MetAPs in the subject being treated. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an antibacterial compound can include a single treatment or can include a series of treatments. It will also be appreciated that the effective dosage of an antibacterial compound used for treatment may increase or decrease over the course of a particular treatment.

Although the precise effects cannot be determined until in vivo tests are performed, it is anticipated that inhibited type-I MetAP enzymes in a treated human patient will dissociate out of the body. Although some decreased methionine cleavage may result from the removal of the type-I enzymes, the remaining type-II enzymes should provide adequate function until the treatment is completed and the body formulates new type-I enzymes in a normal physiological balance.

Pharmaceutical compositions according to the invention may preferably include a pharmaceutically acceptable carrier as described herein below or as generally known in the pharmaceutical arts.

A pharmaceutical composition of the invention is formulated to be compatible with its intended route of administration. Examples of routes of administration include parenteral, e.g., intravenous, intradermal, subcutaneous, oral, inhalation, transdermal (topical), transmucosal, and rectal administration. Solutions or suspensions used for parenteral, intradermal, or subcutaneous application can include, by way of example and not limitation, the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates; and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include, for example, physiological saline, bacteriostatic water, or phosphate buffered saline (PBS). In all cases, the pharmaceutical composition must be sterile and should be fluid to the extent that they can be injected through a syringe. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, or sodium chloride in the composition. Prolonged absorption of the injectable compositions can be brought about by including in the composition an agent which delays absorption, for example, aluminum monostearate or gelatin.

Sterile injectable solutions can be prepared by incorporating the antibacterial compound in the required amount in an appropriate solvent with one or a combination of the ingredients enumerated above, as required, followed by filtered sterilization. Generally, dispersions are prepared by incorporating the antibacterial compound into a sterile vehicle that contains a basic dispersion medium and the required other ingredients from those enumerated above. In the case of sterile powders for the preparation of sterile injectable solutions, the preferred methods of preparation are vacuum drying and freeze-drying which yields a powder of the antibacterial compound plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the antibacterial compound can be incorporated with excipients and used in the form of tablets, troches, or capsules. Oral compositions can also include an enteric coating. Oral compositions can also be prepared using a fluid carrier for use as a mouthwash, wherein the antibacterial compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the antibacterial compounds are delivered in the form of an aerosol spray from a pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the antibacterial compounds are formulated into ointments, salves, gels, or creams as generally known in the art.

The antibacterial compounds can also be prepared in the form of suppositories (e.g., with conventional suppository bases such as cocoa butter and other glycerides) or retention enemas for rectal delivery.

In one embodiment, the antibacterial compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

The antibacterial compounds of the invention can also be incorporated into pharmaceutical compositions that allow for the sustained delivery of the antibacterial compounds to a subject for a period of at least several weeks to a month or more.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of antibacterial compounds calculated to produce the desired therapeutic effect in association with the required pharmaceutical carrier. The specification for the dosage unit forms of the invention are dictated by and directly dependent on the unique characteristics of the antibacterial compound and the particular therapeutic effect to be achieved, and the limitations inherent in the art of compounding such antibacterial compounds for the treatment of individuals.

Toxicity and therapeutic efficacy of such antibacterial compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Antibacterial compounds which exhibit large therapeutic indices are preferred. While antibacterial compounds that exhibit toxic side effects may be used, care should be taken to design a delivery system that targets such antibacterial compounds to the site of affected tissue in order to minimize potential damage to uninfected cells and, thereby, reduce side effects.

The data obtained from the cell culture assays and animal studies can be used in formulating a range of dosage for use in humans. The dosage of such antibacterial compounds lies preferably within a range of circulating concentrations that include the ED50 with little or no toxicity. The dosage may vary within this range depending upon the dosage form employed and the route of administration utilized. For any antibacterial compounds used in the methods of the invention, the therapeutically effective dose can be estimated initially from cell culture assays. A dose may be formulated in animal models to achieve a circulating plasma concentration range that includes the IC50 (i.e., the concentration of the antibacterial compound which achieves a half-maximal inhibition of symptoms) as determined in cell culture. Such information can be used to more accurately determine useful doses in humans. Levels in plasma may be measured, for example, by high performance liquid chromatography.

The following examples are given to illustrate the present invention, and are not intended to limit the scope of the invention.

### EXAMPLES

As illustrated in the following examples, the antibacterial activity of the compounds of the present invention against bacterial pathogens is demonstrated by the compounds' ability to inhibit growth of defined strains of human or animal pathogens.

### Examples 1-3

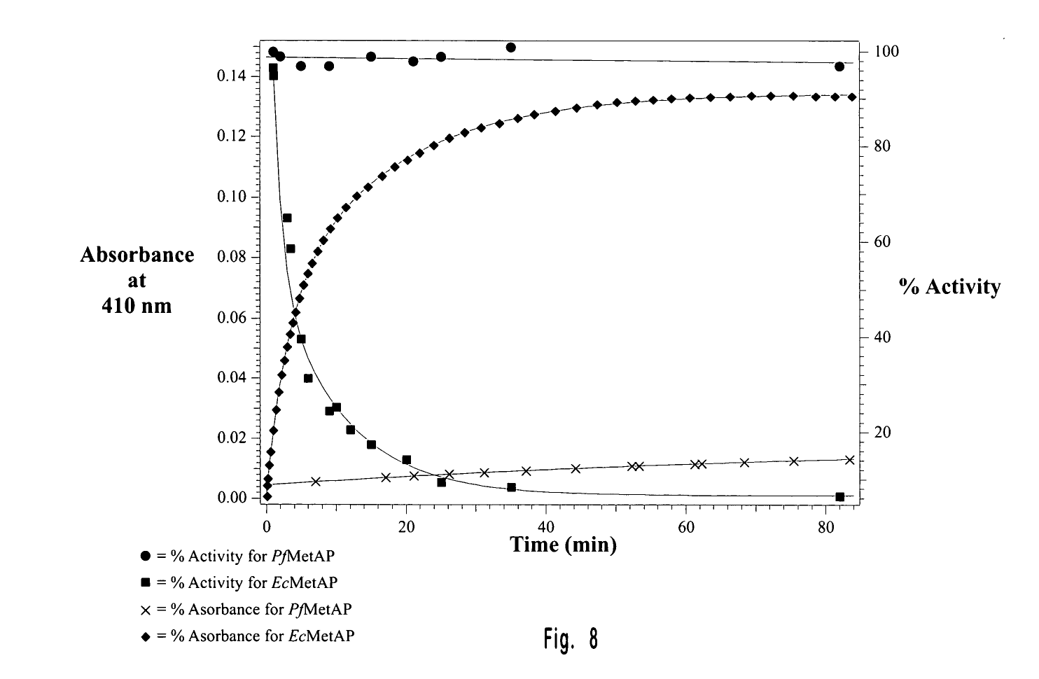

In order to determine if Cys59 and Cys70 are involved in substrate recognition and binding, it was examined if the cysteine-specific chemical modification reagent, 5,5′-dithio-bis(2-nitrobenzoic acid) (DTNB) inhibits the hydrolysis of methionylprolyl-p-nitroanilide (MP-p-NA) by EcMetAP. A 200 μM solution of DTNB was prepared in 50 mM HEPES buffer, pH 7.5, 150 M KCl and added to a 5 PM sample of Co(II)-loaded EcMetAP-I under strict anaerobic conditions. Upon the addition of DTNB, EcMetAP quickly lost catalytic activity and was completely inactive after only 50 min (FIG. 8).

These data indicate that a cysteine residue is involved in blocking substrate binding. Inactivation of EcMetAP-I by DTNB was found to be dependent on both time and the concentration of EcMetAP-I.

Identical experiments, performed on the type-II MetAP, PjMetAP-II, which does not contain a cysteine residue in the substrate recognition pocket, revealed no inactivation due to DTNB even after several hours, as indicated in FIG. 8.

### Example 2

Similarly, no loss in catalytic activity was observed for the HsMetAP type-II enzyme in the presence of 200 μM DTNB. (Example 3) These data indicate that the cysteine residues in the active site pocket of EcMetAP are involved in substrate recognition and that the covalent attachment of an inhibitor to these cysteine residues blocks the substrates ability to bind to the enzyme. These data further support that the Cys 59 and Cys70 Cys residues in type-I MetAPs can be exploited as targets for antibacterial agents.

### Examples 4-6

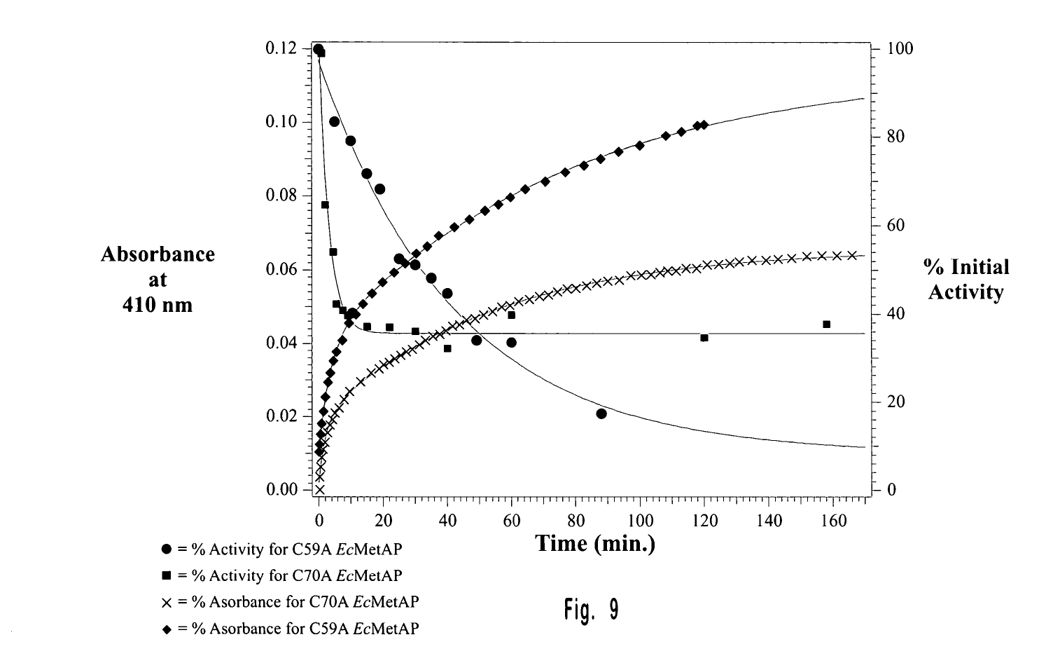

Since at least one surface accessible cysteine residue, in addition to Cys59 and Cys70, exists in EcMetAP-I, reaction of DTNB with other residues cannot be ruled out. In order to circumvent this problem, both the C59A and C70A EcMetAP-I mutant enzymes were prepared and purified using the QuikChange Site-directed Mutagenesis Kit (Stratagene) and each mutation was verified by nucleotide sequencing. Both mutant enzymes bind one equivalent of Co(II) or Fe(II) tightly, based on ICP-AES analysis, which is identical to wild-type EcMetAP, and their kinetic parameters are listed below. Interestingly, k_{cat} does not change significantly for C59A EcMetAP but drops ˜45-fold for the C70A EcMetAP enzyme. Moreover, the Kₘ values for both Co(II)— and Fe(II)-loaded EcMetAP C59A and C70A double and quadruple, respectively. These data support that these two cysteine residues are involved in the substrate recognition and binding events. Reaction of a 10 μM solution of DTNB with C59A EcMetAP under strict anaerobic conditions resulted in the complete inactivation of the catalytic activity albeit at a slower rate (FIG. 9). Surprisingly, the observed inactivation rate for C70A EcMetAP is identical to wild-type enzyme (FIG. 9). These data suggest that C59 is the primary target of DTNB and can be covalently modified.

TABLE 1
Kinetic Constants for Fe(II)- and Co(II)-loaded wild-type, C59A, and
C70A EcMetAP towards MGMM at 30° C. and pH 7.5.

| | Kinetic | Wild- | | |
|---|---|---|---|---|
| Metal | Constants | Type | C59A | C70A |
| Co(II) | Kₘ (mM) | 3.2 ± 0.1 | 6.4 ± 0.2 | 10 ± 0.2 |
| | k_{cat} (s⁻¹) | 19.5 | 12 | 0.7 |
| | k_{cat}/Kₘ (M⁻¹s⁻¹) | 6,100 | 1,875 | 70 |
| | Vₘₐₓ (U/mg) | 39 ± 3 | 24 ± 3 | 1.4 ± 0.5 |
| Fe(II) | Kₘ (mM) | 2.0 ± 0.3 | 8.0 ± 0.2 | 11 ± 0.2 |
| | k_{cat} (s⁻¹) | 28 | 38 | 0.9 |
| | k_{cat}/Kₘ (M⁻¹s⁻¹) | 14,000 | 4,780 | 77 |
| | Vₘₐₓ (U/mg) | 55 ± 3 | 69 ± 2 | 1.7 ± 0.5 |

### Examples 7 and 8

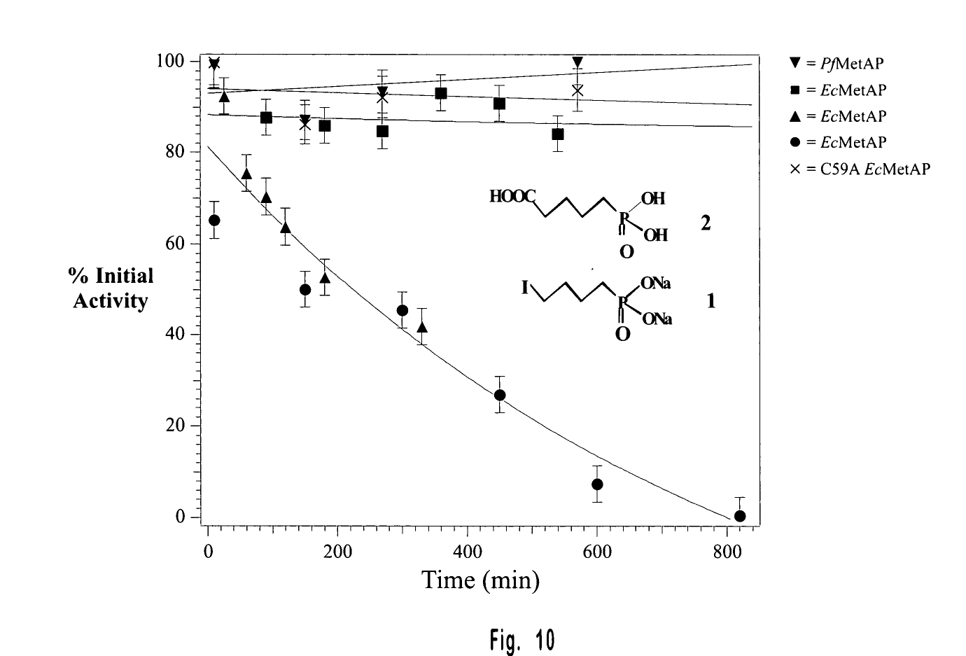

Since only C59 appears to be accessible for covalent modification, two potential inhibitors (Inhibitor 1 and Inhibitor 2, as depicted in FIG. 10) were prepared based on molecular modeling, one of which contains a good leaving group (I⁻) and should be susceptible to nucleophilic attack while the second will not undergo nucleophilic addition. Reaction of wild-type EcMetAP-I with both Inhibitor 1 and Inhibitor 2 for up to 12 hours resulted in the complete and irreversible loss in catalytic activity for EcMetAP-I in the presence of Inhibitor 1 (FIG. 10). As expected, Inhibitor 2 did not significantly alter the catalytic activity of EcMetAP-I. PJMetAP-II was not significantly inhibited by either Inhibitor 1 or Inhibitor 2.

Matrix-Assisted Laser Desorption Ionization-Time of Flight (MALDI-TOF) spectrometric analysis was performed on EcMetAP-I in both the presence and absence of Inhibitor 1. In the absence of Inhibitor 1, a molecular mass 29,500±10 was obtained whereas upon reaction of EcMetAP-I with Inhibitor 1, a small shift in the mass peak was observed suggesting the addition of 140 mass units. These data match well with the calculated mass of Inhibitor 1 after reaction with EcMetAP-I. Therefore, it was determined that Inhibitor 1 is covalently bound to EcMetAP-I via C59.

### Example 9

6-phosphonhexanoic acid (1) and 4-iodobutylphosphonic acid (2) were synthesized in a form of disodium salts. For 4-iodobutylphosphonic acid (2), 4-bromobutylphosphonic acid diethyl ester (3.28 g, 12 mmol) was refluxed with hydroiodic acid (55-58%, 20 mL) for 24 h. The cooled postreaction mixture was diluted with water (100 mL) and extracted with dichlomethane (3×50 mL). The resulting water solution was evaporated under reduced pressure, diluted with water (50 mL) and reevaporated. This procedure was repeated 3 times. The resulting dark red residue, was dissolved in acetone (50 mL) and titrated with concentrated aqueous sodium hydroxide. A white precipitate was filtered off, washed with acetone and diethyl ether and dried in vacuo to give 4-iodobutylphosphonic acid disodium salt (3.70 g, 100%). ¹H NMR (D₂O): 3.15-3.20 (m, 2H), 1.80-1.94 (m, 2H), 1.50-1.70 (m, 4H). 31P NMR (D₂O): 26.7.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

1. A compound comprising: 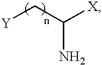
wherein:
X is selected from the group consisting of CH₂SH, CH₂OH, NHOH, PO₃H₂, pyrazoles, imidazoles, oxazoles, isoxazoles, thiazoles, isothiazoles, triazoles, oxadiazoles and thiadiazoles; and
Y is selected from the group consisting of: COCZ, C(EWG)Z, SOCZ, SO₂CZ, 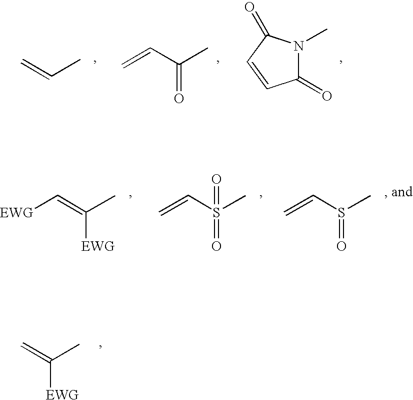
and pharmaceutically acceptable salts thereof, wherein:
EWG is an electron withdrawing group selected from the group consisting of CHO, COR, COOH, COOR, NO₂, CN, SOR, SO₂R, and SO₂OR;
Z is selected from the group consisting of chlorine, bromine, and iodine;
R is an alkyl or aryl group selected from the group consisting of methyl, ethyl, propyl, i-propyl, butyl, s-butyl, t-butyl, phenyl, substituted phenyl, naphthyl, substituted naphthyl; and
n is an integer.

2. A compound as recited in claim 1, wherein n is selected from 4 and 5.

3. A pharmaceutical composition for treating microbial infections in a subject, comprising:
a therapeutically effective amount of an agent wherein the agent is selected from the compounds of claim 1, the agent being capable of altering an aspect of Type-I MetAP activity or structure in the subject so as to result in treatment of the bacterial infection; and
a pharmaceutically acceptable carrier.

4. A pharmaceutical composition for treating bacterial infections in a subject, comprising:
a therapeutically effective amount of an agent wherein the agent is selected from the compounds of claim 1, the agent being capable of altering an aspect of Type-I MetAP activity or structure in the subject so as to result in treatment of the bacterial infection; and
a pharmaceutically acceptable carrier.

5. A compound as recited in claim 4, wherein the subject is a human.

6. A compound as recited in claim 4, wherein the agent does not completely inhibit the activity of Type-II MetAP in the subject but is bactericidal by inhibiting the activity of Type-I MetAP in the subject.

7. A method of providing a dosage of an antibacterial compound to a subject in need thereof, the method comprising administering to the subject an effective amount of a compound as recited in claim 1.

8. A compound comprising a formula selected from the group consisting of: 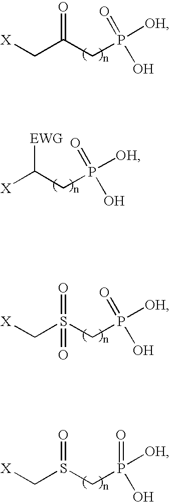
and pharmaceutically acceptable salts thereof, wherein:
X is chlorine, bromine, or iodine;
EWG is an electron withdrawing group selected from the group consisting of CHO, COR, COOH, COOR, NO₂, CN, SOR, SO₂R, and SO₂OR;
R is an alkyl or aryl group selected from the group consisting of methyl, ethyl, propyl, i-propyl, butyl, s-butyl, t-butyl, phenyl, substituted phenyl, naphthyl, and substituted naphthyl; and
n is an integer.

9. A compound as recited in claim 8, wherein n is selected from 4 and 5.

10. A pharmaceutical composition for treating bacterial infections in a subject, comprising:
a therapeutically effective amount of an agent wherein the agent is selected from the compounds of claim 8, the agent being capable of altering an aspect of Type-I MetAP activity or structure in the subject so as to result in treatment of the bacterial infection; and
a pharmaceutically acceptable carrier.

11. A compound as recited in claim 10, wherein the subject is a human.

12. A compound as recited in claim 10, wherein the agent does not completely inhibit the activity of Type-II MetAP in the subject but is bactericidal by inhibiting the activity of Type-I MetAP.

13. A method of providing a dosage of an antibacterial compound to a subject in need thereof, the method comprising administering to the subject an effective amount of a compound as recited in claim 8.

14. A compound comprising a formula selected from the group consisting of: 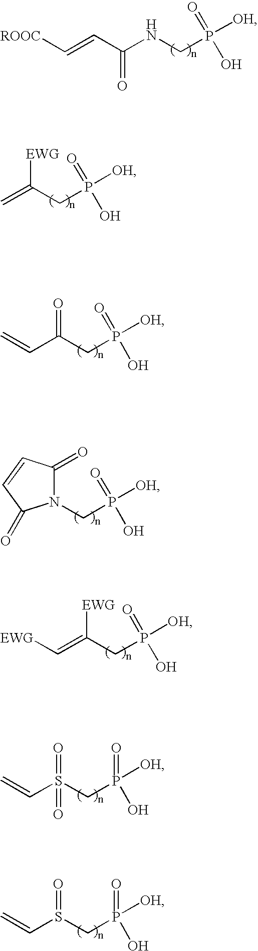
and pharmaceutically acceptable salts thereof, wherein:
EWG is an electron withdrawing group selected from the group consisting of CHO, COR, COOH, COOR, NO₂, CN, SOR, SO₂R, and SO₂OR;
R is an alkyl or aryl group selected from the group consisting of methyl, ethyl, propyl, i-propyl, butyl, s-butyl, t-butyl, phenyl, substituted phenyl, naphthyl, substituted naphthyl; and
n is an integer.

15. A compound as recited in claim 14, wherein n is selected from 4 and 5.

16. A pharmaceutical composition for treating bacterial infections in a subject, comprising:
a therapeutically effective amount of an agent wherein the agent is selected from the compounds of claim 14, the agent being capable of altering an aspect of Type-I MetAP activity or structure in the subject so as to result in treatment of the bacterial infection; and
a pharmaceutically acceptable carrier.

17. A compound as recited in claim 16, wherein the subject is a human.

18. A compound as recited in claim 16, wherein the agent does not completely inhibit the activity of Type-II MetAP in the subject but is bactericidal by inhibiting the activity of Type-I MetAP.

19. A method of providing a dosage of an antibacterial compound to a subject in need thereof, the method comprising administering to the subject an effective amount of a compound as recited in claim 14.

20. A method of providing an antibacterial dosage to a subject in need thereof which comprises:
administering to a subject an effective amount of a compound that is selectively configured to inhibit Type-I MetAP, the compound comprising the formula:
A-B-C
wherein:
A is a functional group selected to covalently bond with a recognition site on Type-I MetAP;
C is an electrophilic functional group selected to inhibit a catalytic site on Type-I MetAP; and
B is a series of groups selected to separate A and C such that each of A and C effectively bind to the respective recognition and active sites on Type-I MetAP;
and pharmaceutically acceptable salts thereof.

21. A method as recited in claim 20, wherein A comprises 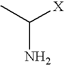
wherein X is selected from the group consisting of CH₂SH, CH₂OH, NHOH, PO₃H₂, pyrazoles, imidazoles, oxazoles, isoxazoles, thiazoles, isothiazoles, triazoles, oxadiazoles and thiadiazoles.

22. A method as recited in claim 20, wherein B comprises a four or five carbon chain.

23. A method as recited in claim 20, wherein C is selected from the group consisting of: COCZ, C(EWG)Z, SO₂CZ, 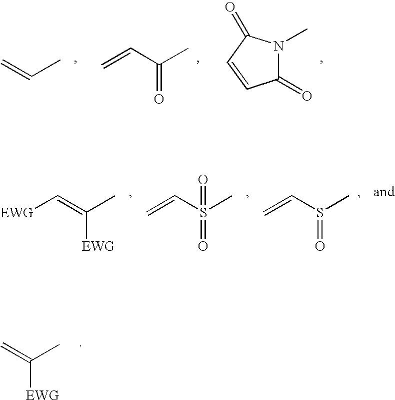
wherein:
EWG is an electron withdrawing group selected from the group consisting of CHO, COR, COOH, COOR, NO₂, CN, SOR, SO₂R, and SO₂OR;
Z is selected from the group consisting of chlorine, bromine, and iodine; and
R is an alkyl or aryl group selected from the group consisting of methyl, ethyl, propyl, i-propyl, butyl, s-butyl, t-butyl, phenyl, substituted phenyl, naphthyl, substituted naphthyl.

24. A method as recited in claim 20, wherein the antibacterial dosage further comprises a pharmaceutically acceptable carrier.

25. A method as recited in claim 20, wherein the compound does not completely inhibit the activity of Type-II MetAP in the subject but is bactericidal by inhibiting the activity of Type-I MetAP in the subject.
